(19) United States
(12) Patent Application Publication
(10) Pub. No.: US 2025/0331742 A1
(43) Pub. Date: Oct. 30, 2025

(54) ANTENNA SYSTEM FOR ANALYTE SENSOR SYSTEM

(71) Applicant: Dexcom, Inc., San Diego, CA (US)

(72) Inventors: Amit VERMA, Dania Beach, FL (US); Terry T. THOM, Coral Springs, FL (US)

(21) Appl. No.: 19/186,322

(22) Filed: Apr. 22, 2025

Related U.S. Application Data
(60) Provisional application No. 63/638,890, filed on Apr. 25, 2024.

(51) Int. Cl. A61B 5/145; A61B 5/00; A61B 90/00; H01Q 7/00; H05K 1/02

(52) U.S. Cl. CPC A61B 5/14532; A61B 5/14503; A61B 5/6801; A61B 90/06; H01Q 7/00; H05K 1/0243; A61B 2090/064; A61B 2560/0462; A61B 2562/0219; A61B 2562/0271; A61B 2562/166; A61B 2562/227

(57) ABSTRACT

Aspects of the present disclosure provide an analyte sensor system. The analyte sensor system may include an analyte sensor configured to measure analyte levels of the user, a stamp antenna configured to transmit data indicative of the measured analyte levels, a printed circuit board (PCB) that operatively connects the analyte sensor to the stamp antenna, and a housing that encases at least the stamp antenna, the PCB, and a first portion of the analyte sensor. The housing may have a bottom portion through which a second portion of the analyte sensor protrudes to an exterior of the housing of the analyte sensor system. The bottom portion of the housing may be configured to be attached to a body of the user. The stamp antenna may be disposed on a bottom side of the PCB facing the bottom portion of the housing.

### CROSS-REFERENCE TO RELATED APPLICATIONS

This application claims priority to and benefit of U.S. Provisional Patent Application No. 63/638,890, filed Apr. 25, 2024, which is hereby assigned to the assignee hereof and hereby expressly incorporated by reference herein in its entirety as if fully set forth below and for all applicable purposes.

### TECHNICAL FIELD

The present disclosure relates generally to an electronic device, such as an analyte sensor system for monitoring analyte values of a user.

### BACKGROUND

Diabetes is a metabolic condition relating to the production or use of insulin by the body. Insulin is a hormone that allows the body to use glucose for energy, or store glucose as fat. When a person eats a meal that contains carbohydrates, the food is processed by the digestive system, which produces glucose in the person's blood. Blood glucose may be used for energy or stored as fat. The body normally maintains blood glucose levels in a range that provides sufficient energy to support bodily functions and avoids problems that may arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels may elevate beyond normal ranges. The state of having a higher-than-normal blood sugar level is called “hyperglycemia.” Chronic hyperglycemia may lead to several of health problems, such as cardiovascular disease, cataract and other eye problems, nerve damage (neuropathy), and kidney damage. Hyperglycemia may also lead to acute problems, such as diabetic ketoacidosis—a state in which the body becomes excessively acidic due to the presence of blood glucose and ketones, which are produced when the body is unable to use glucose. The state of having lower than normal blood glucose levels is called “hypoglycemia.” Severe hypoglycemia may lead to acute crises that may result in seizures or death.

A diabetes patient may receive insulin to manage blood glucose levels. Insulin may be received, for example, through a manual injection with a needle. Wearable insulin pumps are also available. Diet and exercise also affect blood glucose levels.

Diabetes conditions are sometimes referred to as “Type 1” and “Type 2”. A Type 1 diabetes patient is typically able to use insulin when it is present, but the body is unable to produce adequate insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

### SUMMARY

One aspect of the present disclosure provides an analyte sensor system. The analyte sensor system may include an analyte sensor configured to measure analyte levels of a user of the analyte sensor system; a stamp antenna configured to transmit data indicative of the measured analyte levels; a printed circuit board (PCB) that operatively connects the analyte sensor to the stamp antenna for transmission of the data indicative of the measured analyte levels; and a housing that encases at least the stamp antenna, the PCB, and a first portion of the analyte sensor, wherein: the housing has a bottom portion through which a second portion of the analyte sensor protrudes to an exterior of the housing of the analyte sensor system; the bottom portion of the housing is configured to be attached to a body of the user; and the stamp antenna is coupled to a bottom side of the PCB facing the bottom portion of the housing.

Another aspect provides a method for wireless communication by an analyte sensor system. The method includes obtaining, from an analyte sensor of the analyte sensor system, electrical current indicative of analyte levels of a user of the analyte sensor system; processing the electrical current to generate data indicative of the analyte levels; and transmitting the data indicative of the analyte levels using a stamp antenna, wherein the stamp antenna is disposed on a bottom side of printed circuit board (PCB) of the analyte sensor system configured to face a body of the user when the analyte sensor system is worn by the user.

Another aspect provides an apparatus for wireless communication by an analyte sensor system. The apparatus includes one or more processors configured to execute instructions stored on one or more memories and to cause the analyte sensor system to: obtain, from an analyte sensor of the analyte sensor system, electrical current indicative of analyte levels of a user of the analyte sensor system; process the electrical current to generate data indicative of the analyte levels; and transmit the data indicative of the analyte levels using a stamp antenna, wherein the stamp antenna is disposed on a bottom side of printed circuit board (PCB) of the analyte sensor system configured to face a body of the user when the analyte sensor system is worn by the user.

Another aspect provides an antenna system of an analyte sensor system for communicating data indicative of measured analyte levels of a user of the analyte sensor system. The antenna system includes a stamp antenna configured to transmit the data indicative of the measured analyte levels; a ground plane configured to: obtain energy radiated from the stamp antenna including the data indicative of the measured analyte levels; and re-radiate the energy, including the data indicative of the measured analyte levels, away from the body of the user; and an exciter device configured to couple the energy radiated from the stamp antenna to the ground plane, wherein: the stamp antenna is coupled to a bottom side of a printed circuit board (PCB); the bottom side of the PCB is configured to face a body of the user when the analyte sensor system is worn by the user; and the ground plane and the exciter device are disposed on a top side of the PCB opposite to the bottom side of the PCB.

### BRIEF DESCRIPTION OF THE DRAWINGS

Further aspects of the present disclosure will be more readily appreciated upon review of the detailed description of the various disclosed embodiments, described below, when taken in conjunction with the accompanying figures.

FIG. 1 illustrates aspects of an example analyte monitoring system that may be used in connection with some embodiments.

FIG. 2 illustrates aspects of an example health monitoring and management system that may be used in connection with some embodiments.

FIG. 3A is an example analyte sensor system, in accordance with some embodiments.

FIG. 3B is an example analyte sensor system, in accordance with some embodiments.

FIG. 4 illustrates aspects of an example analyte sensor system, in accordance with some embodiments.

FIG. 5 illustrates aspects of an example analyte sensor system, in accordance with some embodiments.

FIG. 6 illustrates a cross section of an analyte sensor system, in accordance with some embodiments.

FIGS. 7A, 7B, and 7C illustrate various views of an analyte sensor system including a stamp antenna that may be used for communicating with a display device or other partner device, in accordance with some embodiments.

FIG. 8 includes a simplified circuit diagram illustrating how certain components of an analyte sensor system may be electrically connected, in accordance with some embodiments.

FIG. 9 illustrates a method for wireless communication by an analyte sensor system, in accordance with some embodiments.

The figures, described in greater detail in the description and examples below, are provided for purposes of illustration only, and merely depict typical or example embodiments of the disclosure. The figures are not intended to be exhaustive or to limit the disclosure to the precise form disclosed. It should also be understood that the disclosure may be practiced with modification or alteration, and that the disclosure may be limited only by the claims and the equivalents thereof.

### DETAILED DESCRIPTION

Aspects of the present disclosure provide systems, methods, and devices for improving a communication range of an analyte sensor system. An analyte sensor system may be worn by a user and is configured to continuously monitor analyte levels of the user. The data indicating these analyte levels may then be transmitted from the analyte sensor system to a display device (e.g., smart phone) using one or more antennas, allowing the user to conveniently track their analyte levels. Certain existing analyte sensor systems are bulky and tend to be uncomfortable to wear. As a result, there is a constant competitive drive to miniaturize analyte sensor systems, for example, to provide better comfort, discreet usage, and/or ease of use to the user.

However, this miniaturization may have negative effects on communication or transmission ranges of the analyte sensor systems, which may result in the display device not receiving the analyte levels of the user. One manner to reduce these negative effects and improve communication and transmission rages of analyte sensor systems may be to use a stamp antenna.

However, the stamp antenna may be required to be placed on a top side of a printed circuit board (PCB) included within an analyte sensor system such that the stamp antenna faces away from a body of a user of the analyte sensor system when the analyte sensor system is worn by the user. For example, by positioning the stamp antenna on the top side of the PCB transmissions by the stamp antenna will be transmitted away from the body of the user rather than being absorbed by the body of the user, which would cause the transmissions to not be properly received by a display device.

However, due to the miniaturization of the analyte sensor system and positioning constraints of an analyte sensor (e.g., a device that is partially implanted in the user and that measures the analyte levels of the user), the PCB may need to be positioned in the analyte sensor adjacent to a top portion of a housing of the analyte sensor system, leaving little to no room for a stamp antenna to be placed on the top side of the PCB. Additionally, while a bottom side of the PCB (e.g., facing the body of the user when the analyte sensor system in worn by the user) may include a sufficient amount of space for the stamp antenna, in this scenario, the stamp antenna would be facing into the body of the user and, as a result, transmissions by the stamp antenna would be absorbed by the body of the user, preventing the transmissions from being properly received by the display device.

Accordingly, aspects of the present disclosure provide techniques for equipping an analyte sensor system with a stamp antenna and using the stamp antenna to provide better antenna efficiency and communication range for the analyte sensor system. For example, to overcome the space restrictions and positioning constraints related to the analyte sensor discussed above, these techniques may involve positioning the stamp antenna on a bottom side of a PCB of the analyte sensor system facing in a direction towards a body of a user of the analyte sensor system. Further, to overcome the negative effects discussed above relating to placing the stamp sensor on the bottom side of the PCB (e.g., transmissions being absorbed by the body of the user and not being properly received by the display device), the techniques presented herein further involve the use of an exciter device positioned on a top side of the PCB. For example, the exciter device may be used to cause energy radiated from the stamp antenna to couple with, and be re-radiated by, a ground plane on the top side of the PCB, effectively reversing a radiation pattern of the stamp antenna from being directed into the body of the user to being directed away from the body of the user and allowing transmissions from the stamp antenna to be received by the display device.

The details of some example embodiments of the systems, methods, and devices of the present disclosure are set forth in this description and in some cases, in other portions of the disclosure. Other features, objects, and advantages of the disclosure will be apparent to one of skill in the art upon examination of the present disclosure, description, figures, examples, and claims. It is intended that all such additional systems, methods, devices, features, and advantages be included within this description (whether explicitly or by reference), be within the scope of the present disclosure, and be protected by one or more of the accompanying claims.

### System Overview and Example Configurations

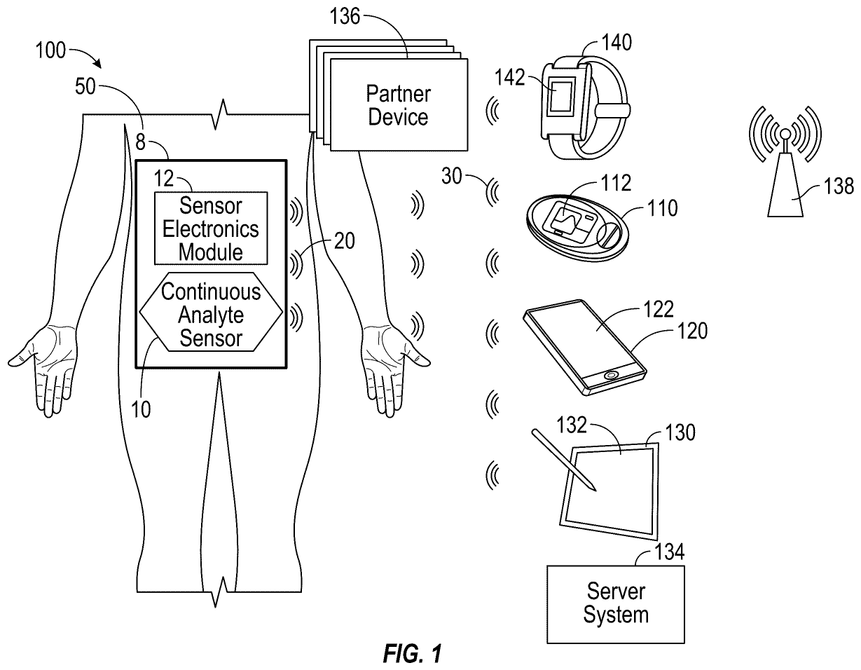

FIG. 1 depicts an analyte monitoring system 100 that may be used in connection with embodiments of the present disclosure that involve gathering, monitoring, and/or providing information regarding analyte values present in a user's body, including for example the user's blood glucose values, other analytes, multiple multiplexed or simultaneous measured analytes, or the like. System 100 depicts aspects of analyte sensor system 8 that may be communicatively coupled to display devices 110, 120, 130, and 140, partner devices 136, and/or server system 134.

Analyte sensor system 8 in the illustrated embodiment includes analyte sensor electronics module 12 and analyte sensor 10 associated with analyte sensor electronics module 12. Analyte sensor electronics module 12 may be electrically and mechanically coupled to analyte sensor 10 before analyte sensor 10 is implanted in a user or host. Accordingly, analyte sensor 10 may not require a user to couple analyte sensor electronics module 12 to analyte sensor 10. For example, analyte sensor electronics module 12 may be physically/mechanically and electrically coupled to analyte sensor 10 during manufacturing, and this physical/mechanical and electrical connection may be maintained during shipping, storage, insertion, use, and removal of analyte sensor system 8. As such, the electro-mechanically connected components (e.g., analyte sensor 10 and analyte sensor electronics module 12) of analyte sensor system 8 may be referred to as a “pre-connected” system. Analyte sensor electronics module 12 may be in wireless communication (e.g., directly or indirectly) with one or more of display devices 110, 120, 130, and 140. In addition, or alternatively to display devices 110, 120, 130, and 140, analyte sensor electronics module 12 may be in wireless communication (e.g., directly or indirectly) with partner devices 136 and/or server system 134. Likewise, in some examples, display devices 110-140 may additionally or alternatively be in wireless communication (e.g., directly or indirectly) with partner devices 136 and/or server system 134. Various couplings shown in FIG. 1 may be facilitated with wireless access point (WAP) 138, as also mentioned below.

In certain embodiments, analyte sensor electronics module 12 includes electronic circuitry associated with measuring and processing analyte sensor data or information, including prospective algorithms associated with processing and/or calibration of the analyte sensor data/information. Analyte sensor electronics module 12 may be physically/mechanically connected to analyte sensor 10 and may be integral with (non-releasably attached to) or releasably attachable to analyte sensor 10. Analyte sensor electronics module 12 may also be electrically coupled to analyte sensor 10, such that the components may be electromechanically coupled to one another. Analyte sensor electronics module 12 may include hardware, firmware, and/or software that enables measurement and/or estimation of levels of the analyte in a host/user via analyte sensor 10 (e.g., which may be/include a glucose sensor). For example, analyte sensor electronics module 12 may include one or more of a potentiostat, a power source for providing power to analyte sensor 10, other components useful for signal processing and data storage, and a telemetry module for transmitting data from the sensor electronics module to one or more display devices. Electronics may be affixed to a printed circuit board (PCB) within analyte sensor system 8, or platform or the like, and may take a variety of forms. For example, the electronics may take the form of an integrated circuit (IC), such as an Application-Specific Integrated Circuit (ASIC), a microcontroller, a processor, and/or a state machine.

Analyte sensor electronics module 12 may include sensor electronics that are configured to process sensor information, such as sensor data, and generate transformed sensor data and displayable sensor information. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, all of which are incorporated herein by reference in their entireties.

With further reference to FIG. 1 display devices 110, 120, 130, and/or 140 may be configured for displaying (and/or alarming) displayable sensor information that may be transmitted by analyte sensor electronics module 12 (e.g., in a customized data package that is transmitted to the display devices based on their respective preferences). Each of display devices 110, 120, 130, or 140 may (respectively) include a display such as touchscreen display 112, 122, 132, /or 142 for displaying sensor information and/or analyte data to a user and/or receiving inputs from the user. For example, a graphical user interface (GUI) may be presented to the user for such purposes. In embodiments, the display devices may include other types of user interfaces such as voice user interface instead of or in addition to a touchscreen display for communicating sensor information to the user of the display device and/or receiving user inputs. In embodiments, one, some, or all of display devices 110, 120, 130, 140 may be configured to display or otherwise communicate the sensor information as it is communicated from analyte sensor electronics module 12 (e.g., in a data package that is transmitted to respective display devices), without any additional prospective processing required for calibration and/or real-time display of the sensor data.

The plurality of display devices 110, 120, 130, 140 depicted in FIG. 1 may include a custom display device, for example, analyte display device 110, specially designed for displaying certain types of displayable sensor information associated with analyte data received from analyte sensor electronics module 12 (e.g., a numerical value and/or an arrow, in embodiments). In embodiments, one of the plurality of display devices 110, 120, 130, 140 includes a smartphone, such as a mobile phone, based on an Android, IOS, or other operating system, and configured to display a graphical representation of the continuous sensor data (e.g., including current and/or historic data).

As further illustrated in FIG. 1 and mentioned above, analyte monitoring system 100 may also include WAP 138 that may be used to couple one or more of analyte sensor system 8, the plurality display devices 110, 120, 130, 140 etc., server system 134, and partner devices 136 to one another. For example, WAP 138 may provide WiFi and/or cellular or other wireless connectivity within analyte monitoring system 100. Near Field Communication (NFC) may also be used among devices of analyte monitoring system 100 for exchanging data, as well as for performing specialized functions, e.g., waking up or powering a device or causing the device (e.g., analyte sensor electronics module 12 and/or a transmitter) to exit a lower power mode or otherwise change states and/or enter an operational mode. Server system 134 may be used to collect analyte data from analyte sensor system 8 and/or the plurality of display devices, for example, to perform analytics thereon, generate universal or individualized models for glucose levels and profiles, provide services or feedback, including from individuals or systems remotely monitoring the analyte data, and so on.

Partner device(s) 136, by way of overview and example, may usually communicate (e.g., wirelessly) with analyte sensor system 8, including for authentication of partner device(s) 136 and/or analyte sensor system 8, as well as for the exchange of analyte data, medicament data, other data, and/or control signaling or the like. Partner devices 136 may include a passive device in example embodiments of the disclosure. One example of partner device 136 may be an insulin pump for administering insulin to a user in response and/or according to an analyte level of the user as measured/approximated using analyte sensor system 8. For a variety of reasons, it may be desirable for such an insulin pump to receive and track glucose values transmitted from analyte sensor system 8 (with reference to FIG. 1 for example). One example reason for this is to provide the insulin pump a capability to suspend/activate/control insulin administration to the user based on the user's glucose value being below/above a threshold value.

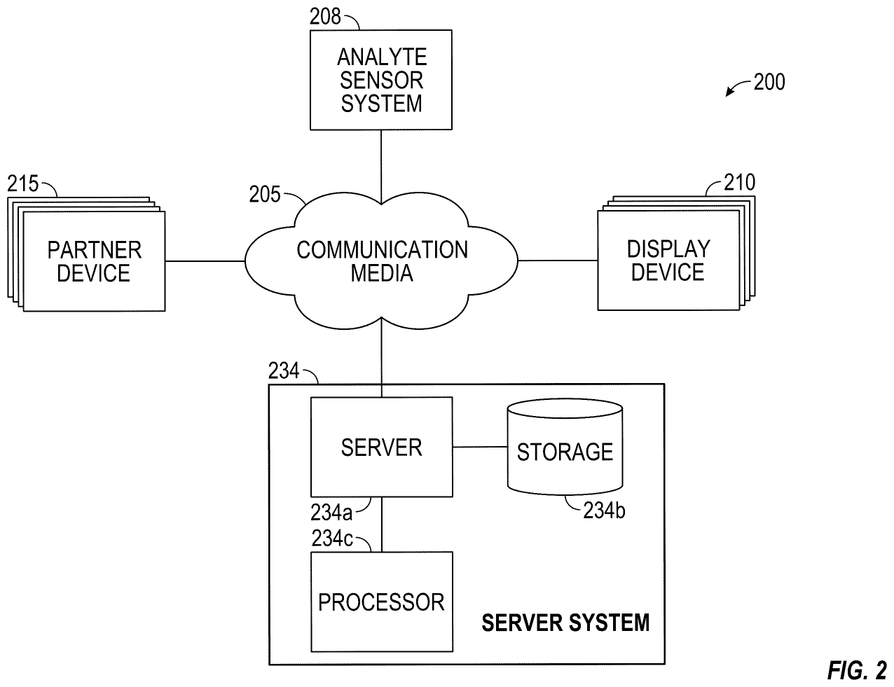

Referring now to FIG. 2 a health monitoring and management system 200 is depicted. The health monitoring and management system 200 may be used in connection with implementing embodiments of the disclosed systems, methods, apparatuses, and/or devices, including, for example, aspects described above in connection with FIG. 1 By way of example, various below-described components of FIG. 2 may be used to provide wireless communication of analyte (e.g., glucose) data, for example among/between analyte sensor system 208, display devices 210, partner devices 215, and/or one or more server systems 234, and so on. In some cases, analyte sensor system 208 illustrated in FIG. 2 may be an example of the analyte sensor system 8 illustrated in FIG. 1. Additionally, in some cases, the display devices 210 illustrated in FIG. 2 may be examples of the display devices 110, 120, 130, and 140 illustrated in FIG. 1. Additionally, in some cases, partner devices 215 illustrated in FIG. 2 may be examples of the partner device 136 illustrated in FIG. 1.

As shown in FIG. 2 the health monitoring and management system 200 may include analyte sensor system 208, one or more display devices 210, and/or one or more partner devices 215. Additionally, in the illustrated embodiment, the health monitoring and management system 200 includes server system 234, which may in turn include server 234a coupled to processor 234c and storage 234b. Analyte sensor system 208 may be coupled to display devices 210, partner devices 215, and/or server system 234 via communication media 205. Some details of the processing, gathering, and exchanging of data, and/or executing actions (e.g., providing medicaments or related instructions) by analyte sensor system 208, partner devices 215, and/or display device 210, etc., are provided below. Herein, display devices 210, partner devices 215, and server system 234 may be referred to as display devices and may be configured to communicate with analyte sensor system 208.

Analyte sensor system 208, display devices 210, and/or partner devices 215 may exchange messaging (e.g., control signaling) via communication media 205, and communication media 205 may also be used to deliver analyte data to display devices 210, partner devices 215, and/or server system 234. As alluded to above, display devices 210 may include a variety of electronic computing devices, such as a smartphone, tablet, laptop, wearable device, etc. Display devices 210 may also include analyte display device 110 that may be customized for the display and conveyance of analyte data and related notifications etc. Partner devices 215 may include medical devices, such as an insulin pump or pen, connectable devices, such as a smart fridge or mirror, key fob, and other devices.

In embodiments, communication media 205 may implemented using one or more wireless communication protocols, such as for example BLUETOOTH, BLUETOOTH LOW Energy (BLE), ZigBee, WiFi, IEEE 802.11 protocols, Infrared (IR), Radio Frequency (RF), 2G, 3G, 4G, 5G, etc., and/or wired protocols and media. It will also be appreciated upon studying the present disclosure that communication media may be implemented as one or more communication links, including in some cases, separate links, between the components of the health monitoring and management system 200, whether or not such links are explicitly shown in FIG. 2 or referred to in connection therewith. By way of illustration, analyte sensor system 208 may be coupled to display device 210 via a first link of communication media 205 using BLE, while analyte sensor system 208 may be coupled to server system 234 by a second link of communication media 205 using a WiFi communication protocol. In embodiments, a BLE signal may be temporarily attenuated to minimize data interceptions. For example, attenuation of a BLE signal through hardware or firmware design may occur temporarily during moments of data exchange (e.g., pairing).

In embodiments, the elements of the health monitoring and management system 200 may be used to perform operations of various processes described herein and/or may be used to execute various operations and/or features described herein with regard to one or more disclosed systems and/or methods. Upon studying the present disclosure, one of skill in the art will appreciate that the health monitoring and management system 200 may include single or multiple analyte sensor systems 208, communication media 205, and/or server systems 234.

As mentioned, communication media 205 may be used to connect or communicatively couple analyte sensor system 208, display devices 210, partner devices 215, and/or server system 234 to one another or to a network. Communication media 205 may be implemented in a variety of forms. For example, communication media 205 may include one or more of an Internet connection, such as a local area network (LAN), a person area network (PAN), a wide area network (WAN), a fiber optic network, internet over power lines, a hard-wired connection (e.g., a bus), DSL, and the like, or any other kind of network connection or communicative coupling. Communication media 205 may be implemented using any combination of routers, cables, modems, switches, fiber optics, wires, radio (e.g., microwave/RF, AM, FM links etc.), and the like. Upon reading the present disclosure, one of skill in the art will recognize other ways to implement communication media 205 for communications purposes and will also recognize that communication media 205 may be used to implement features of the present disclosure using as of yet undeveloped communications protocols that may be deployed in the future.

Further referencing FIG. 2 server 234a may receive, collect, and/or monitor information, including analyte data, medicament data, and related information, from analyte sensor system 208, partner devices 215 and/or display devices 210, such as input responsive to the analyte data or medicament data, or input received in connection with an analyte monitoring application running on analyte sensor system 208 or display device 210, or a medicament delivery application running on display device 210 or partner device 215. As such, server 234a may receive, collect, and/or monitor information from partner devices 215, such as, information related to the provision of medicaments to a user and/or information regarding the operation of one or more partner devices 215. Server 234a may also receive, collect, and/or monitor information regarding a user of analyte sensor system 208, display devices 210, and/or partner devices 215.

In embodiments, server 234a may be adapted to receive such information via communication media 205. This information may be stored in storage 234b and may be processed by processor 234c. For example, processor 234c may include an analytics engine capable of performing analytics on information that server 234a has collected, received, etc. via communication media 205. In embodiments, server 234a, storage 234b, and/or processor 234c may be implemented as a distributed computing network, such as a Hadoop RTM network, or as a relational database or the like. The aforementioned information may then be processed at server 234a such that services may be provided to analyte sensor system 208, display devices 210, partner devices 215, and/or a user(s) thereof. For example, such services may include diabetes management feedback for the user.

Server 234a may include, for example, an Internet server, a router, a desktop or laptop computer, a smartphone, a tablet, a processor, a module, or the like, and may be implemented in various forms, including, for example, an integrated circuit or collection thereof, a printed circuit board or collection thereof, or in a discrete housing/package/rack or multiple of the same. In embodiments, server 234a at least partially directs communications made over communication media 205. Such communications may include the delivery of analyte data, medicament data, and/or messaging related thereto (e.g., advertisement, authentication, command, or other messaging). For example, server 234a may process and exchange messages between and/or among analyte sensor system 208, display devices 210, and/or partner devices 215 related to frequency bands, timing of transmissions, security/encryption, alarms, alerts, notifications, and so on. Server 234a may update information stored on analyte sensor system 208, partner devices 215, and/or display devices 210, for example, by delivering applications thereto or updating the same, and/or by reconfiguring system parameters or other settings of analyte sensor system 208, partner devices 215, and/or display devices 210. Server 234a may send/receive information to/from analyte sensor system 208, partner devices 215, and/or display devices 210 in real time, periodically, sporadically, or on an event-drive basis. Further, server 234a may implement cloud computing capabilities for analyte sensor system 208, partner devices 215, and/or display devices 210.

With the above description of aspects of the presently disclosed systems and methods for wireless communication of analyte data, examples of some specific features of the present disclosure will now be provided. It will be appreciated by one of skill in the art upon studying the present disclosure that these features may be implemented using aspects and/or combinations of aspects of the example configurations described above, whether or not explicit reference is made to the same.

### Analyte Data

Referring back to FIG. 1 as mentioned above, in embodiments, analyte sensor system 8 is provided for measurement of an analyte in a host or user. By way of an overview and an example, analyte sensor system 8 may be implemented as an encapsulated microcontroller that makes sensor measurements, generates analyte data (e.g., by calculating values for continuous glucose monitoring data), and engages in wireless communications (e.g., via Bluetooth and/or other wireless protocols) to send such data to remote devices (e.g., display devices 110, 120, 130, 140, partner devices 136, and/or server system 134).

Analyte sensor system 8 may include: analyte sensor 10 configured to measure a concentration or level of the analyte in the host, and analyte sensor electronics module 12 that is typically physically connected to analyte sensor 10 before analyte sensor 10 is implanted in a user. In some embodiments, the analyte sensor 10 may have a first portion and a second portion. The first portion may be encased in a housing of the analyte sensor system 8 and coupled to the sensor electronics module 12 using a conductive epoxy and an encapsulant layer. The second portion may protrude through the housing of the analyte sensor system and may be implanted in the user. In some embodiments, the analyte sensor 10 may be a single-analyte sensor or a multi-analyte sensor capable of measuring one or more analytes, such as glucose, lactate, potassium, and the like. In embodiments, analyte sensor electronics module 12 includes electronics configured to process a data stream associated with an analyte concentration measured by analyte sensor 10, in order to generate sensor information that includes raw sensor data, transformed sensor data, and/or any other sensor data, for example. Analyte sensor electronics module 12 may further be configured to generate analyte sensor information that is customized for respective display devices 110, 120, 130, 140, partner devices 136, and/or server system 134. Analyte sensor electronics module 12 may further be configured such that different devices may receive different sensor information and may further be configured to wirelessly transmit sensor information to such display devices 110, 120, 130, 140, partner devices 136, and/or server system 134.

The term “analyte” as used herein is a broad term and is to be given its ordinary and customary meaning to a person of ordinary skill in the art (and is not to be limited to a special or customized meaning), and furthermore refers without limitation to a substance or chemical constituent in a biological fluid (for example, blood, interstitial fluid, cerebral spinal fluid, lymph fluid or urine) that may be analyzed. Analytes may include naturally occurring substances, artificial substances, metabolites, and/or reaction products. In some embodiments, the analyte for measurement by the sensor heads, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, M CAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase 1; 17-alpha-hydroxyprogesterone; hypoxanthine phosphoribosyl transferase; immunoreactive trypsin; lactate; lead; lipoproteins ((a), B/A-1,); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferring; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids may also constitute analytes in certain embodiments. The analyte may be naturally present in the biological fluid, for example, a metabolic product, a hormone, an antigen, an antibody, and the like. Alternatively, the analyte may be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body may also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

### Analyte Sensor System

As described to above with reference to FIG. 1 in some embodiments, analyte sensor 10 includes a continuous glucose sensor, for example, a subcutaneous, transdermal (e.g., transcutaneous), or intravascular device. In embodiments, such a sensor or device may continuously measure and analyze glucose measurements in the interstitial fluid, blood samples, etc., depending on whether the device is subcutaneous, transdermal, or intravascular. Analyte sensor 10 may use any method of analyte measurement, including for example glucose-measurement, including enzymatic, chemical, physical, electrochemical, spectrophotometric, polarimetric, calorimetric, iontophoretic, radiometric, immunochemical, and the like.

In embodiments where analyte sensor 10 is a glucose sensor, analyte sensor 10 may use any method, including invasive, minimally invasive, and non-invasive sensing techniques (e.g., fluorescence monitoring), or the like, to provide a data stream indicative of the concentration of glucose in a host. The data stream may be a raw data signal, which may be converted into a calibrated and/or filtered data stream that may be used to provide a useful value of glucose to a user, such as a patient or a caretaker (e.g., a parent, a relative, a guardian, a teacher, a doctor, a nurse, or any other individual that has an interest in the wellbeing of the host).

A glucose sensor may be any device capable of measuring the concentration of glucose. According to one example embodiment described below, an implantable glucose sensor may be used. However, it should be understood that the devices and methods described herein may be applied to any device capable of detecting a concentration of an analyte, glucose for example, and providing an output signal that represents the concentration of the analyte, again glucose for example (e.g., as a form of analyte data).

In embodiments, analyte sensor 10 is an implantable glucose sensor, such as described with reference to U.S. Pat. No. 6,001,067 and U.S. Patent Publication No. US-2005-0027463-A1. In embodiments, analyte sensor 10 is a transcutaneous glucose sensor, such as described with reference to U.S. Patent Publication No. US-2006-0020187-A1. In embodiments, analyte sensor 10 is configured to be implanted in a host vessel or extracorporeally, such as is described in U.S. Patent Publication No. US-2007-0027385-A1, co-pending U.S. Patent Publication No. US-2008-0119703-A1 filed Oct. 4, 2006, U.S. Patent Publication No. US-2008-0108942-A1 filed on Mar. 26, 2007, and U.S. Patent Application No. US-2007-0197890-A1 filed on Feb. 14, 2007. In embodiments, the continuous glucose sensor includes a transcutaneous sensor such as described in U.S. Pat. No. 6,565,509 to Say et al., for example. In embodiments, analyte sensor 10 is a continuous glucose sensor that includes a subcutaneous sensor such as described with reference to U.S. Pat. No. 6,579,690 to Bonnecaze et al. or U.S. Pat. No. 6,484,046 to Say et al., for example. In embodiments, the continuous glucose sensor includes a refillable subcutaneous sensor such as described with reference to U.S. Pat. No. 6,512,939 to Colvin et al., for example. The continuous glucose sensor may include an intravascular sensor such as described with reference to U.S. Pat. No. 6,477,395 to Schulman et al., for example. The continuous glucose sensor may include an intravascular sensor such as described with reference to U.S. Pat. No. 6,424,847 to Mastrototaro et al., for example.

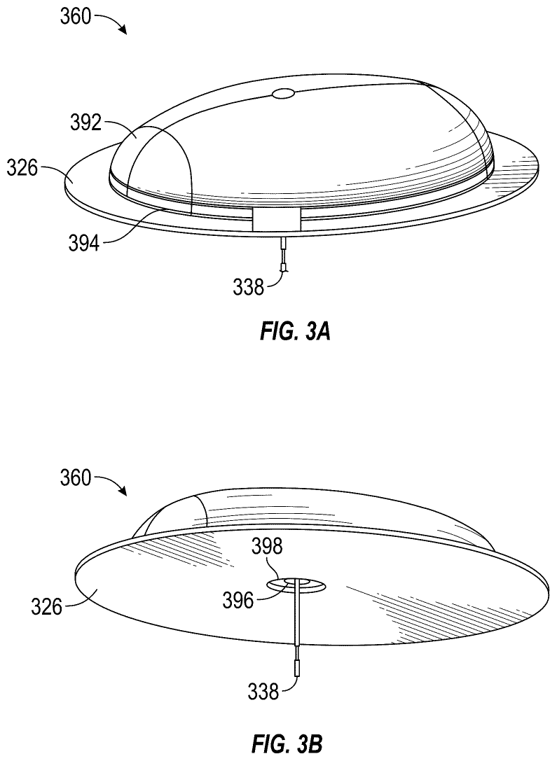

FIG. 3A illustrates a perspective view of an on-skin sensor assembly 360 that may be used in connection with the analyte sensor system 8 of FIG. 1 and/or the analyte sensor system 208 of FIG. 2. For example, on-skin sensor assembly 360 may be or include analyte sensor system 8 and/or analyte sensor system 208. On-skin sensor assembly 360 may include an outer housing with a first, top portion 392 and a second, bottom portion 394. In embodiments, the outer housing may include a clamshell design. On-skin sensor assembly 360 may include, for example, similar components as analyte sensor electronics module 12 described above in connection with FIG. 1 for example, a potentiostat, a power source for providing power to analyte sensor 10, signal processing components, data storage components, and a communication module (e.g., a telemetry module) for one-way or two-way data communication, a printed circuit board (PCB), an integrated circuit (IC), an Application-Specific Integrated Circuit (ASIC), a microcontroller, and/or a processor.

As shown in FIG. 3A, the outer housing may feature a generally oblong shape. The outer housing may further include aperture 396 disposed substantially through a center portion of outer housing and adapted for sensor 338 and needle insertion through a bottom of on-skin sensor assembly 360. In embodiments, aperture 396 may be a channel or elongated slot. On-skin sensor assembly 360 may further include an adhesive patch 326 configured to secure on-skin sensor assembly 360 to skin of the host. In embodiments, adhesive patch 326 may include an adhesive suitable for skin adhesion, for example a pressure sensitive adhesive (e.g., acrylic, rubber-based, or other suitable type) bonded to a carrier substrate (e.g., spun lace polyester, polyurethane film, or other suitable type) for skin attachment, though any suitable type of adhesive is also contemplated. As shown, adhesive patch 326 may feature an aperture 398 aligned with aperture 396 such that sensor 338 may pass through a bottom of on-skin sensor assembly 360 and through adhesive patch 326.

FIG. 3B illustrates a bottom perspective view of on-skin sensor assembly 360 of FIG. 3A. FIG. 3B further illustrates aperture 396 disposed substantially in a center portion of a bottom of on-skin sensor assembly 360, and aperture 398, both adapted for sensor 338 and needle insertion.

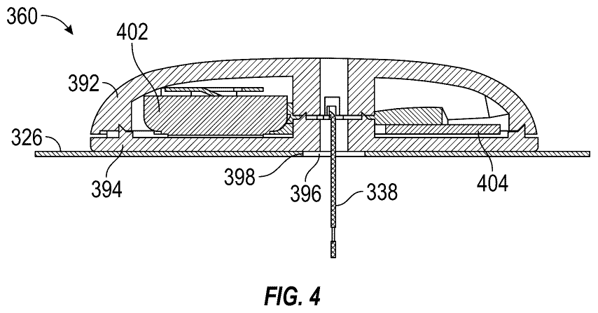

FIG. 4 illustrates a cross-sectional view of on-skin sensor assembly 360 of FIGS. 3A and 3B. FIG. 4 illustrates first, top portion 392 and second, bottom portion 394 of the outer housing, adhesive patch 326, aperture 396 in the center portion of on-skin sensor assembly 360, aperture 398 in the center portion of adhesive patch 326, and sensor 338 passing through aperture 396. The electronics unit, previously described in connection with FIG. 3A, may further include circuit board 404 and battery 402 configured to provide power to at least circuit board 404.

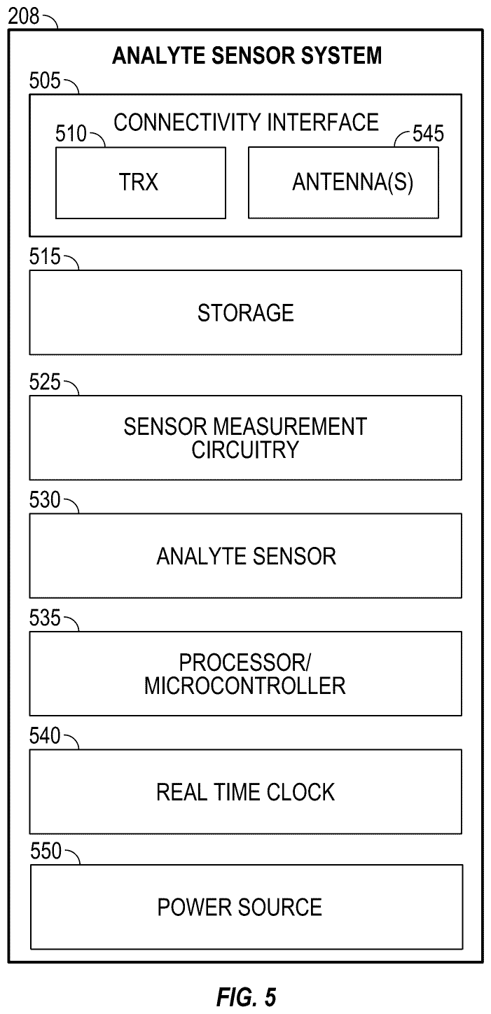

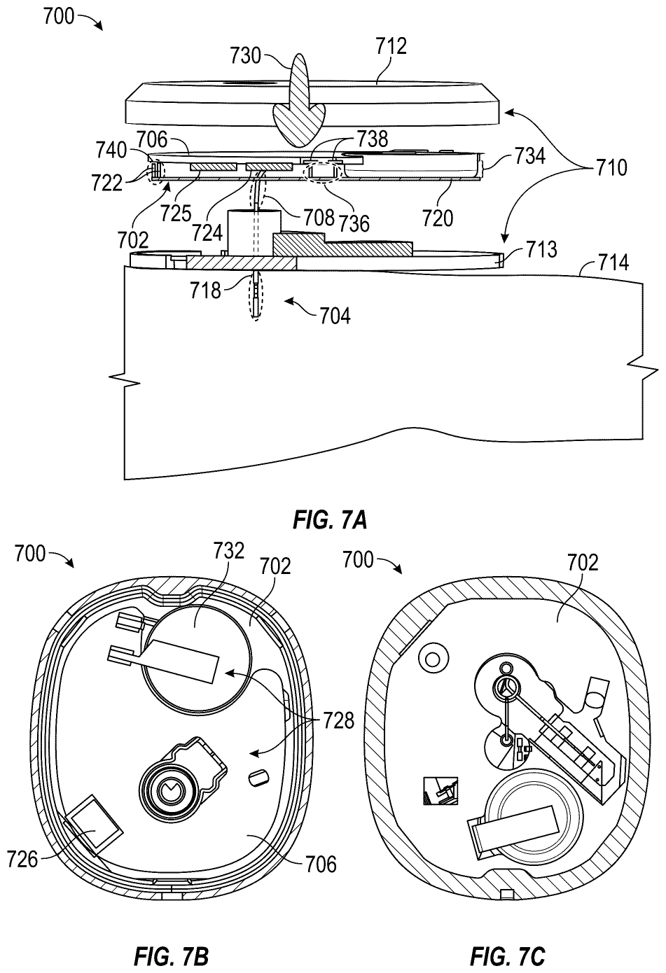

Turning now to FIG. 5, a more detailed functional block diagram of analyte sensor system 208 (discussed above, for example, in connection with FIGS. 1 and 2) is provided. As noted above, the analyte sensor system 208 may be an example of the analyte sensor system 8 illustrated in FIG. 1. As shown in FIG. 5, analyte sensor system 208 may include an analyte sensor 530 (e.g., which may be an example of the analyte sensor 10 illustrated in FIG. 1) coupled to sensor measurement circuitry 525 on a printed circuit board (PCB) for processing and managing sensor data, obtained from the analyte sensor 530, indicative of analyte levels of a user of the analyte sensor system 208. In some embodiments, the analyte sensor 530 may be an example of the analyte sensor 704 depicted and described with respect to FIGS. 7A, 7B, and 7C. For example, the analyte sensor 530 may include at least a first portion that may be encased in a housing of the analyte sensor system 208 and coupled to the sensor measurement circuitry 525 on the PCB using a conductive epoxy and an encapsulant layer. The analyte sensor 530 may also include a second portion that may protrude through the housing of the analyte sensor system 208 and may be implanted under the skin of a user when the analyte sensor system 208 is being worn by the user.

Sensor measurement circuitry 525 may be coupled to processor/microcontroller 535 (e.g., which may be part of analyte sensor electronics module 12 in FIG. 1). In some embodiments, processor/microcontroller 535 may perform part or all of the functions of sensor measurement circuitry 525 for obtaining and processing sensor measurement values from the analyte sensor 530 and generating analyte data representative of the sensor measurement values.

Processor/microcontroller 535 may be further coupled to a radio unit or transceiver 510 (e.g., which may be part of analyte sensor electronics module 12 in FIG. 1). In some embodiments, the processor/microcontroller 535 may be configured to provide sending sensor data, such as the analyte data, and other data to the transceiver 510 for transmission to an external device, such as display device 210 (referencing FIG. 2 by way of example). The transceiver 510 may also be configured to receive, from the external device, control information including requests for certain information and commands to perform certain actions. In some cases, the transceiver 510 may include logic or circuitry for communicating (e.g., transmitting and receiving) using different communication protocols, such as BLUETOOTH, BLUETOOTH Low Energy (BLE), near-field communication (NFC), WiFi, Third Generation Partnership Project (3GPP)-based wireless communication protocols, and other wireless communication protocols. In some embodiments, the transceiver 510 may be coupled to an antenna system 545 associated with the connectivity interface 505, allowing the analyte sensor system 208 to wirelessly transmit and receive data. For example, the transceiver 510 may be configured to output data, such as the analyte data for wireless transmission via one or more antennas of the antenna system 545 or may be configured to obtain data that is wirelessly received via the one or more antennas of the antenna system 545. In some cases, the one or more antennas of the antenna system 545 may include a stamp antenna, such as the stamp antenna 702 depicted and described with respect to FIGS. 7A, 7B, and 7C. In some cases, the antenna system 545 may be tuned to a particular frequency depending on a communication protocol used for communicating data. For example, in some embodiments, the antenna system 545 may include one or more antennas tuned for communicating data via a BLE protocol (e.g., tuned to 2.4 gigahertz). In some embodiments, the antenna system 545 may include one or more antennas tuned for communicating data via an NFC protocol (e.g., tuned to 13.56 megahertz).

Analyte sensor system 208, in example implementations, gathers analyte data using the analyte sensor 530 and transmits the same or a derivative thereof to display device 310, partner device 315, and/or server system 334 using the transceiver 510 and antenna system 545. Data points regarding analyte values may be gathered and transmitted over the life of the analyte sensor 530. New measurements and/or related information may be transmitted often enough for a remote device/individual to adequately monitor analyte (e.g., glucose) levels.

It is to be appreciated that some details of the processing, gathering, and exchanging data by analyte sensor system 208, partner devices 315, and/or display device 310 etc. are provided elsewhere herein. It will be appreciated upon studying the present disclosure that analyte sensor system 208 may contain several like components that are described with respect to FIG. 1 or 2 at least for some embodiments herein. The details and uses of such like components may therefore be understood vis-a-vis analyte sensor system 208 even if not expressly described here with reference to FIG. 5.

### Aspects Related to a Reverse Stamp Antenna for an Analyte Sensor System

Patients with diabetes may benefit from real-time diabetes management guidance that is determined based on a physiological state of the patient. In certain cases, the physiological state of the patient may be determined using diagnostics systems, such as an analyte sensor system (e.g., analyte sensor system 8 and/or analyte sensor system 208). For example, in some embodiments, analyte sensor system 208 may be configured to measure analyte levels of a user and provide the measured analyte levels to display device for display to the user. The display device may be configured to inform the user about the identification and/or prediction of adverse events based on the measured analyte levels, such as hyperglycemia and hypoglycemia. In some embodiments, the analyte sensor system may be configured to identify and/or predict these adverse events and provide information indicative of these adverse events to the display device for display to the user.

For example, the analyte sensor system 208 of FIG. 5 may be worn by a patient and configured to continuously measure the user's analyte levels over time using a continuous analyte sensor, such as the analyte sensor 530. The measured analyte levels may then be processed by the analyte sensor system 208 (e.g., by the processor/microcontroller 535) to generate data indicative of the measured analyte levels, which may be output by the processor/microcontroller 535 to the transceiver 510 of the analyte sensor system 208 for wireless transmission to a communications device, such as one or more of display devices 110, 120, 130, 140 depicted and described with respect to FIG. 1 and/or the display device 210 depicted and described with respect to FIG. 2. In some embodiments, the analyte data may be transmitted to the communications device using one or more antennas of the antenna system 545 and a particular wireless communication protocol, such as BLUETOOTH, BLE, NFC, WiFi, 3GPP-based wireless communication protocols, or other wireless communication protocols.

Typical analyte sensor systems include a sensor electronics module that houses sensor electronics. For example, a housing of the sensor electronics module may have a thickness of about 7 millimeters (mm), allowing the one or more antennas of these analyte sensor systems to be positioned at a distance of about 5.5 mm away from a user's body due to certain design constraints. However, there is a constant competitive drive to miniaturize these analyte sensor systems to provide better comfort, discreet usage, and/or ease of use to the user. As a result, a next-generation analyte sensor system may be designed to have a thickness of less than half of current analyte sensor systems. For example, the next-generation analyte sensor system may have a thickness of about 2.9 mm. This reduction in thickness of the next-generation analyte sensor system may result in the distance between the one or more antennas and the user's body being reduced to about 2.2 mm.

This reduction in distance between the one or more antennas and the user's body may cause bandwidth issues associated with the one or more antennas of the analyte sensor system. For example, as the one or more antennas are positioned closer to the user's body, power radiated from the one or more antennas (e.g., from a back lobe) may be absorbed by the user's body, causing a variation of input impedance, frequency shift and reduced efficiency and gain of the one or more antennas. As a result, due to the miniaturization of the next-generation analyte sensor system, the user's body may negatively affect a radiation pattern of the one or more antennas, causing a reduction in antenna efficiency and communication range between the next-generation analyte sensor system and associated communications devices (e.g., display devices, pumps, etc.) and leading to poor user experience.

One manner to improve antenna efficiency and a communication range of an analyte sensor system may be to use a stamp antenna. Stamp antennas are relatively low cost and may be fabricated using a stamping process in which an antenna is stamped out of a sheet of conductive material. Stamp antennas have a relatively omnidirectional radiation pattern, a mixed polarization, and a high radiation efficiency. Moreover, these antennas are ideal for protocols including Bluetooth®, Wi-Fi, cellular, 2G, 3G, 4G (LTE, NB-IoT), and positioning/GNSS.

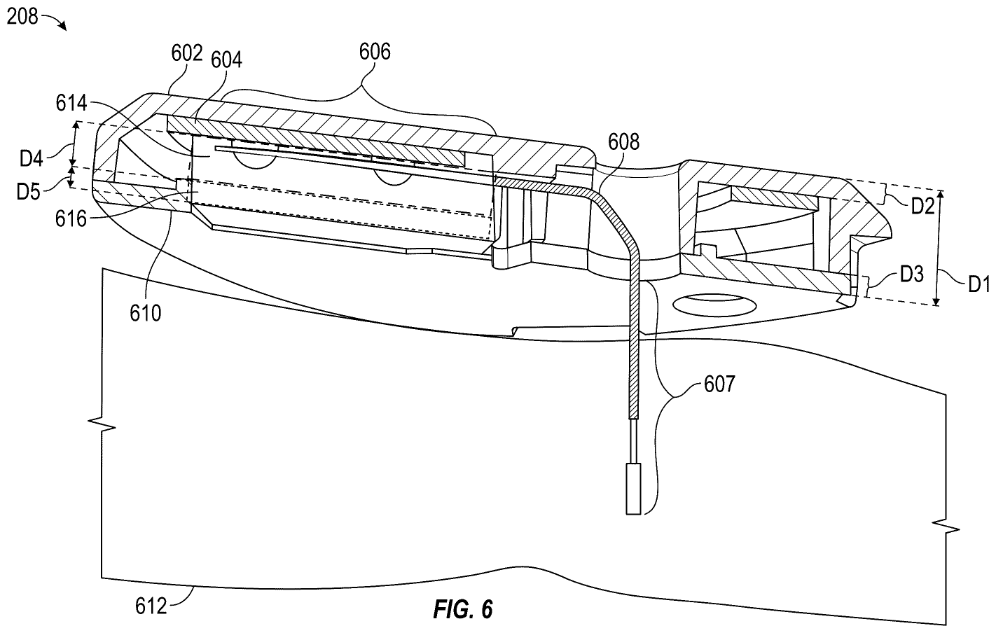

While the use of a stamp antenna may improve antenna efficiency and communication range of the analyte sensor system, the stamp antenna may need to be oriented away from the body of the user of the analyte sensor system to ensure that transmissions by the stamp antenna are properly received by a remotely located display device. However, due to space restrictions within a housing of the analyte sensor system and due to positioning constraints associated with an analyte sensor of the analyte sensor system, it may not be feasible to implement a stamp antenna within the analyte sensor system. An example of these space restrictions and positioning constraints is illustrated in FIG. 6.

For example, FIG. 6 illustrates a cross section of the analyte sensor system 208. As shown, the analyte sensor system 208 includes a housing having a top portion 602 and a bottom portion 610. As shown, the housing of the analyte sensor system 208 may have a total height or thickness (D1) of about 2.9 mm with the top portion 602 having a height or thickness (D2) of about 0.5 mm and the bottom portion 610 having a height or thickness (D3) of about 0.5 mm. As shown, the bottom portion 610 is configured to be attached to a body 612 of a user (e.g., using an adhesive patch or some other manner of attachment) when the analyte sensor system 208 is being worn by the user. Further, as shown, the top portion 602 is configured to face away from the body 612 of the user. More generally, the top portion 602 is configured to face in a direction opposite to the bottom portion 610.

As shown, the housing of the analyte sensor system 208 encases a PCB 604 and a first portion 606 of an analyte sensor 608 that is electrically connected to the PCB 604. Additionally, as shown, the analyte sensor 608 includes a second portion 607 configured to protrude through the bottom portion 610 of the housing and be implanted in the body 612 of the user. In order to ensure successful attachment of the analyte sensor 608 to the PCB 604 and to ensure proper bend relief for the analyte sensor, the PCB 604 may be required to be positioned at least 1.5 mm above a bottom portion 610 of the analyte sensor system facing the body 612 of the user. The requirement that the PCB 604 be positioned at least 1.5 mm above the bottom portion 610 may be due to a conductive epoxy layer 614 that surrounds and electrically connects the analyte sensor 608 to the PCB 604 as well as an encapsulant layer 616 that surrounds and protects the connection. For example, as shown, the epoxy layer 614 has a maximum height, labeled D4 in FIG. 6, of 1 mm while the encapsulant layer 616 has a height, labeled D5 in FIG. 6, of about 0.5 mm.

Accordingly, as may be seen in FIG. 6, the positioning of the PCB 604 relative to the bottom portion 610 of the analyte sensor system 208 leaves little to no room for a stamp antenna to be positioned on a top side of the PCB 604 facing away from the body 612 of the user since the analyte sensor system 208 is only 2.9 mm thick itself. For example, due to the space needed for the conductive epoxy layer 614 and encapsulant layer 616 (e.g., 1.5 mm), the thickness of the bottom portion 610 (e.g., 0.5 mm), the thickness of the top portion 602 (e.g., 0.5 mm), and the total thickness of the analyte sensor system 208 (e.g., 2.9 mm), the PCB 604 is positioned adjacent to the top portion 602 of the housing of the analyte sensor system 208. As such, this configuration leaves little to no room for a stamp antenna (e.g., having a typical thickness or height of about 1.4 mm) to be positioned on the top side of the PCB 604 that is adjacent to the top portion 602 of the housing.

Additionally, it should be appreciated that placement of the stamp antenna on the top side of the PCB 604 that faces away from the body 612 is desirable since this would result in a radiation pattern of the stamp antenna also facing away from the body 612. More specifically, because the radiation pattern of the stamp antenna is directed away from the body 612, transmissions from the stamp antenna radiate outward (e.g., away from the body 612), allowing these transmissions to be properly received by a display device. However, due to the space restrictions and positioning constraints discussed above, placing the stamp antenna on the top side of the PCB 604 may not be an option. Another option may be to place the stamp antenna on a bottom side of the PCB 604 facing the body 612. However, in this scenario, the radiation pattern of the stamp antenna would be facing into the body 612, resulting in transmission from the stamp antenna being absorbed by the body 612 and not being properly received by the display device.

Accordingly, aspects of the present disclosure provide techniques for equipping an analyte sensor system with a stamp antenna and using the stamp antenna to provide better antenna efficiency and communication range for the analyte sensor system. For example, to overcome the space restrictions and analyte sensor placement constraints discussed above, these techniques may involve positioning the stamp antenna on a bottom side of a PCB of the analyte sensor system facing in a direction towards a body of a user of the analyte sensor system. Further, to overcome the negative effects discussed above relating to placing the stamp sensor on the bottom side of the PCB (e.g., transmissions being absorbed by the body of the user and not being properly received by a display device), the techniques presented herein further involve the use of an exciter device positioned on a top side of the PCB. For example, the exciter device may be used to cause energy radiated from the stamp antenna to couple with, and be re-radiated by, a ground plane on the top side of the PCB, effectively reversing a radiation pattern of the stamp antenna from being directed into the body of the user to being directed away from the body of the user and allowing transmissions from the stamp antenna to be received by a display device.

FIGS. 7A, 7B, and 7C illustrate various views of an analyte sensor system 700 including a stamp antenna that may be used for communicating with a display device or other partner device. For example, FIG. 7A shows an exploded side view of the analyte sensor system 700. FIG. 7B shows a top down view of a PCB included within the analyte sensor system 700 and FIG. 7C shows a bottom up view of the PCB included within the analyte sensor system. It should be appreciated that the analyte sensor system 700 may be an example of the analyte sensor system 8 depicted and described with respect to FIG. 1 and/or the analyte sensor system 208 depicted and described with respect to at least FIGS. 2, 5, and 6.

As shown in FIG. 7A, the analyte sensor system 700 includes an analyte sensor 704 configured to be implanted in a body 714 of a user of the analyte sensor system 700 and to measure analyte levels of the user. Additionally, as shown in FIGS. 7A, 7B, and 7C, the analyte sensor system 700 includes an antenna system comprising at least a stamp antenna 702 configured to transmit data indicative of the measured analyte levels, for example, to a display device. The analyte sensor system 700 also includes a PCB 706 that operatively connects the analyte sensor 704 to the stamp antenna 702 for transmission of the data indicative of the measured analyte levels. Further, as shown, the analyte sensor system 700 includes a housing that encases at least the stamp antenna 702, the PCB 706, and a first portion 708 of the analyte sensor 704. In some embodiments, the first portion 708 of the analyte sensor 704 may be electrically coupled to the PCB 706 using a conductive epoxy and an encapsulant layer, such as the conductive epoxy layer 614 and the encapsulant layer 616 described with respect to FIG. 6.

In some embodiments, the stamp antenna 702 may be any shape that is able to fit within the housing of the analyte sensor system 700 and may be made of various conductive materials, such as copper, aluminum, steel, etc. Additionally, in some embodiments, the stamp antenna 702 may be made from a solid conductive material or may be made of a conductive mesh material.

As shown, the housing 710 includes a top portion 712 that is configured to face away from the body 714 of the user when the analyte sensor system 700 is worn by the user. Additionally, as shown the housing 710 includes a bottom portion 716 through which a second portion 718 of the analyte sensor 704 protrudes to an exterior of the housing 710 of the analyte sensor system 700. In some embodiments, the second portion 718 of the analyte sensor 704 may be configured to be implanted into the body 714 of the user when the analyte sensor system 700 is worn by the user. Additionally, as shown, the bottom portion 716 is configured to face the body 714 of the user when the analyte sensor system 700 is worn by the user. For example, when being worn by the user, the bottom portion 716 may be attached to the body 714 of the user using an adhesive patch or another attachment means.

As discussed above with respect to FIG. 6, due to space restrictions and positioning constraints, the stamp antenna 702 may be coupled to a bottom side of the PCB 706 facing the bottom portion 713 of the housing 710. As shown, the stamp antenna 702 includes a main body portion 720 and plurality of risers 722 that may be used to couple the stamp antenna 702 to the PCB 706 and to provide support and stability to the main body portion 720 of the stamp antenna 702.

Additionally, as shown, the analyte sensor system 700 includes a transceiver 724 and one or more processors 725. In some embodiments, the transceiver 724 may be capable of outputting and receiving signals according to a particular wireless communication standard or protocol, such as Bluetooth, Bluetooth Low Energy, WiFi, or the like. The one or more processors 725 may be configured to obtain the measured analyte levels (e.g., electrical signals or current indicative of the analyte levels) from the analyte sensor 704, process the measured analyte levels (e.g., process the electrical signals or current) to generate the data indicative of the measured analyte levels, and to output the data indicative of the measured analyte levels to the transceiver 724 for transmission via the stamp antenna 702. For example, in some embodiments, stamp antenna 702 may be configured to obtain the data indicative of the measured analyte levels from the transceiver 724 of the analyte sensor system 700. Further, in order to transmit the data indicative of the measured analyte levels, the stamp antenna 702 may be configured to radiate energy including the data indicative of the measured analyte levels.

Additionally, the antenna system of the analyte sensor system 700 may include one or more loop antennas (not shown) for data communication via near field communication (NFC) or radio frequency identification (RFID). For example, analyte sensor system 700 may be configured to wirelessly pair with one or more display devices using NFC when the analyte sensor system 700 and the one or more display devices are proximate to each other. In such example, pairing information, authentication and validation information related to the analyte sensor system and/or display devices may be exchanged during NFC wireless communication using the one or more loop antennas. In some examples, the one or more loop antennas may be used for communicating analyte data. For example, the one or more display devices may initiate NFC communication for analyte data (e.g., may transmit a request to the analyte sensor system 700 for the analyte data via NFC), and in response the analyte sensor system 700 may provide a requested analyte data via NFC to the one or more display devices. It will be appreciated that the display devices may be similarly equipped with such an antenna system for NFC communication. It will be further appreciated that the one or more loop antennas in the analyte sensor system 700 may be disposed on, within, or beneath the PCB 706.

In some examples, the stamp antenna 702 may operate in tandem with the one or more loop antennas used for NFC communication. For example, NFC communication via the one or more loop antennas may be used between the one or more display devices and the analyte sensor system 700 to set up Bluetooth wireless communication via the stamp antenna 702. In such example, once the Bluetooth wireless communication is set up, periodic transmission of the analyte data communication may take place between the analyte sensor system 700 and one or more display devices via at least the stamp antenna 702 using Bluetooth wireless technology. In some embodiments, the analyte sensor system 700 may receive a request for analyte data from the one or more display devices via the one or more loop antennas using NFC communication. Further, in some embodiments, the analyte sensor system 700 may respond to the request by sending the analyte data using Bluetooth wireless communication via the stamp antenna 702.

As discussed above, because the stamp antenna 702 is positioned on the bottom side of the PCB 706 facing the body 714 of the user, transmissions by the stamp antenna 702 would normally be radiated into the body 714, which would prevent or significantly hinder a display device's ability to receive the data indicative of the measured analyte levels from the analyte sensor system 700. To avoid or at least reduce this issue, the antenna system of the analyte sensor system 700 may also include an exciter device 726 disposed on a top side of the PCB 706 (e.g., opposite of the bottom side of the PCB 706) and a ground plane 728 disposed on the top side of the PCB 706, as shown in FIG. 7B. In some embodiments, the ground plane 728 may be a solid ground plane (e.g., composed of a solid area of conductive material, such as copper) or may be a mesh ground plane (e.g., composed of an area of a web-like conductive material).

In some embodiments, the stamp antenna 702 may include a first antenna feed that may be electrically connected to a radio frequency (RF) input/output of the transceiver for receiving and transmitting signals, such as the data indicative of the analyte levels associated with the user of the analyte sensor system 700. Additionally, the stamp antenna 702 may include a second antenna feed that is electrically connected to the exciter device 726 positioned on the top side of the PCB 706 facing away from the body 714 of the user. When transmitting signals (e.g., the data indicative of the analyte levels) using the stamp antenna 702, a voltage may be applied to the exciter device 726 (e.g., by the analyte sensor system 700). The voltage applied to the exciter device 726 may cause a radiation pattern of the stamp antenna 702 to reverse, switching from being directed towards the body 714 of a user of the analyte sensor system to being directed away from the body 714 of the user, as shown at 730 in FIG. 7A.

For example, when not using the exciter device, signals output by the transceiver 724 and transmitted using the stamp antenna 702 would normally be radiated in a direction of the body 714 of the user since the stamp antenna 702 is positioned on the bottom side of the PCB 706 facing the body of the user. However, when the voltage is applied to the exciter device 726, the radiation pattern of the stamp antenna 702 may be reversed and may instead be oriented in a direction away from the body 714 of the user. This may result in the energy radiated by the stamp antenna 702 (e.g., when transmitting the data indicative of the analyte levels of the user) coupling to the ground plane 728 on the top side of the PCB 706 and being re-radiated by the ground plane 728 away from the body 714 of the user. In other words, when transmitting the data indicative of the measured analyte levels, the ground plane 728 is configured to obtain the energy radiated from the stamp antenna through coupling based on the exciter device 726. In some embodiments, based on the exciter device 726, a majority of the energy radiated in a main lobe of the stamp antenna 702 may be radiated away from the body 714 of the user in a direction of the ground plane 728 disposed on the top side of the PCB 706. After obtaining the energy from the stamp antenna 702 through coupling, the ground plane 728 may then re-radiate the energy including the data indicative of the measured analyte levels away from the body 714 of the user.

In some embodiments, as shown in FIG. 7B, the ground plane 728 may occupy a majority of an area of the top side of the PCB 706, except for a portion of the top side dedicated to the exciter device 726. Additionally, in some embodiments, as shown in FIGS. 7A and 7B the analyte sensor system 700 may include a power source 732, such as a battery, that may be positioned in such a manner that a ground terminal of the power source 732 is facing in a same direction of the top side of the PCB 706 and the ground plane 728, thereby effectively extending an area of the ground plane 728 on the top side of the PCB 706. In other words, the ground plane 728 may effectively cover the majority of the area of the top side of the PCB 706 as well as an area associated with the ground terminal of the power source 732.

As noted above, in some embodiments, the stamp antenna 702 may include the plurality of risers 722 that may be used to couple the stamp antenna 702 to the bottom side of the PCB 706 and to provide stability for the stamp antenna 702. As shown in FIG. 7A, the plurality of risers 722 may be configured to raise the main body portion 720 of the stamp antenna 702 off of the bottom side of the PCB 706 by a certain distance 734, forming an area between the main body portion 720 of the stamp antenna 702 and the bottom side of the PCB 706. In some embodiments, the certain distance 734 and resulting area formed between the main body portion 720 of the stamp antenna 702 and the bottom side of the PCB 706 may permit one or more other hardware components to be positioned on the PCB 706 under the stamp antenna, which results in more efficient use of real estate on the PCB 706. For example, in some embodiments, the analyte sensor may include one or more other hardware components, such as the one or more processors 725, the transceiver 724, one or more memories, a modem (e.g., Bluetooth, BLE, WiFi, cellular, etc.), an accelerometer, a force sensor, a temperature sensor, resistors, capacitors, inductors, etc. In such embodiments, the one or more other hardware components may be disposed on the bottom side of the PCB 706 in the area between the main body portion 720 of the stamp antenna 702 and the bottom side of the PCB 706, which utilizes the real estate on the PCB 706 more efficiently.

In some embodiments, a first set of risers 736 of the plurality of risers may be coupled to a first set of floating point pads 738 disposed on (e.g., soldered to) the bottom side of the PCB 706. The first set of risers 736 and the first set of floating point pads 738 may be used to control a radiation pattern of the stamp antenna 702 and how far energy may be radiated by the stamp antenna 702. For example, the first set of risers 736 and the first set of floating point pads 738 may be configured to shape a radiation pattern of the stamp antenna 702. Further, in some embodiments, a second set of risers 740 may be used to provide stability and structural support for the stamp antenna by extending away from the main body portion 720 of the stamp antenna 702 and touching the PCB 706. However, the second set of risers 740 may not be electrically connected to the PCB 706. In other words, the second set of risers 740 may adjoin the PCB 706 but are not electrically coupled to the PCB 706. Because the one or more risers are not electrically connected to the PCB 706, the one or more risers may not significantly affect the radiation pattern of the stamp antenna 702.

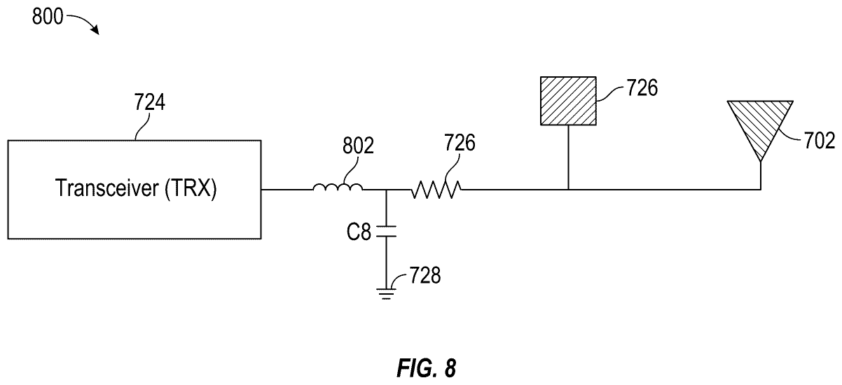

FIG. 8 includes a simplified circuit diagram 800 illustrating how certain components of the analyte sensor system 700 may be electrically connected. For example, as shown, output signals (e.g., the data indicative of the analyte levels) from the transceiver 724 may first pass through an inductor 802 and a resistor 804 before being provided to a first circuit branch including the stamp antenna 702 and the exciter device 726 for transmission. As shown, the stamp antenna 702 and the exciter device 726 are connected in parallel in the first circuit branch. Additionally, as shown, the first circuit branch (e.g., including the stamp antenna 702 and the exciter device 726) may be connected in parallel with a second circuit branch including a capacitor 806 and the ground plane 728.

### Example Operations of an Analyte Sensor System

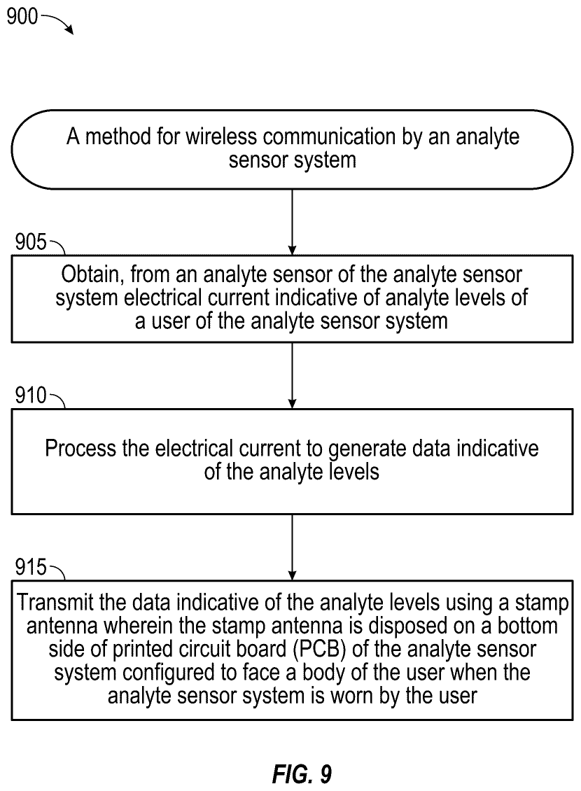

FIG. 9 shows an example of a method 900 for wireless communication by an analyte sensor system, such as the analyte sensor system 208 depicted and described with respect to FIGS. 2, 5, and 6 and/or the analyte sensor system 700 depicted and described with respect to FIG. 7. In some embodiments, method 900 may be performed by one or more processors of the analyte sensor system, such as the processor/microcontroller 535 and/or the one or more processors 725, based on instructions stored in one or more memories. For example, in some embodiments, the analyte sensor system may include one or more memories (e.g., storage 515) including instructions that, when executed by the one or more processors, cause the analyte sensor system to perform the method 900.

Method 900 begins at step 905 with the analyte sensor system obtaining, from an analyte sensor (e.g., analyte sensor 530, 704) of the analyte sensor system, electrical current indicative of analyte levels of a user of the analyte sensor system.

At 910, the analyte sensor system processes the electrical current to generate data indicative of the analyte levels.

At 915, the analyte sensor system transmits the data indicative of the analyte levels using a stamp antenna (e.g., stamp antenna 702). In some embodiments, the stamp antenna is disposed on a bottom side of a printed circuit board (PCB) (e.g., PCB 706) of the analyte sensor system configured to face a body (e.g., body 714) of the user when the analyte sensor system is worn by the user.

In some embodiments, method 900 may further include, when transmitting the data indicative of the analyte levels using the stamp antenna, applying a voltage to an exciter device (e.g., exciter device 726) disposed on a top side of the analyte sensor system configured to face away from the body of the user when the analyte sensor system is worn by the user.

In some embodiments, transmitting the data indicative of the analyte levels using the stamp antenna comprises radiating energy from the stamp antenna including the data indicative of the analyte levels.

In some embodiments, the analyte sensor system further includes a ground plane (e.g., ground plane 728) disposed on the top side of the PCB.

In some embodiments, applying the voltage to the exciter device when transmitting the data indicative of the analyte levels causes the energy radiated from the stamp antenna to couple with the ground plane disposed on the top side of the PCB and to be re-radiated by the ground plane away from the body of the user.

In some embodiments, applying the voltage to the exciter device when transmitting the data indicative of the analyte levels causes a majority of the energy radiated in a main lobe of the stamp antenna to be radiated away from the body of the user in a direction of the ground plane disposed on the top side of the PCB.

### EXAMPLE CLAUSES

Implementation examples are described in the following numbered clauses:

Clause 1: An analyte sensor system, comprising: an analyte sensor configured to measure analyte levels of a user of the analyte sensor system; a stamp antenna configured to transmit data indicative of the measured analyte levels; a printed circuit board (PCB) that operatively connects the analyte sensor to the stamp antenna for transmission of the data indicative of the measured analyte levels; and a housing that encases at least the stamp antenna, the PCB, and a first portion of the analyte sensor, wherein: the housing has a bottom portion through which a second portion of the analyte sensor protrudes to an exterior of the housing of the analyte sensor system; the bottom portion of the housing is configured to be attached to a body of the user; and the stamp antenna is disposed on a bottom side of the PCB facing the bottom portion of the housing.

Clause 2: The analyte sensor system of Clause 1, further comprising an exciter device disposed on a top side of the PCB opposite to the bottom side of the PCB.

Clause 3: The analyte sensor system of Clause 2, further comprising a ground plane disposed on the top side of the PCB.

Clause 4: The analyte sensor system of Clause 3, further comprising: a transceiver; and one or more processors configured to: obtain the measured analyte levels from the analyte sensor; and output the data indicative of the measured analyte levels to the transceiver for transmission via the stamp antenna.

Clause 5: The analyte sensor system of Clause 4, wherein: the stamp antenna is further configured to obtain the data indicative of the measured analyte levels from the transceiver of the analyte sensor system; and in order to transmit the data indicative of the measured analyte levels, the stamp antenna is configured to radiate energy including the data indicative of the measured analyte levels.

Clause 6: The analyte sensor system of Clause 5, wherein the exciter device is configured to couple the radiated energy from the stamp antenna to the ground plane.

Clause 7: The analyte sensor system of Clause 6, wherein, based on the exciter device, the ground plane is configured to: obtain the energy from the stamp antenna; and re-radiate the energy including the data indicative of the measured analyte levels away from the body of the user.

Clause 8: The analyte sensor system of any one of Clauses 5-7, wherein the exciter device is configured to cause a majority of the energy radiated in a main lobe of the stamp antenna to be radiated away from the body of the user in a direction of the ground plane disposed on the top side of the PCB.

Clause 9: The analyte sensor system of any one of Clauses 1-8, wherein: the first portion of the analyte sensor is electrically coupled to the PCB using a conductive epoxy and an encapsulant layer; and the second portion of the analyte sensor is configured to be implanted into the body of the user.

Clause 10: The analyte sensor system of any one of Clauses 1-9, wherein: the stamp antenna includes a main body portion and plurality of risers coupled to the main body portion; and the plurality of risers are configured to raise the main body portion of the stamp antenna off of the bottom side of the PCB by a certain distance and form an area between the main body portion of the stamp antenna and the bottom side of the PCB.

Clause 11: The analyte sensor system of Clause 10, wherein: a first set of risers are electrically coupled to a first set of floating point pads disposed on the bottom side of the PCB; and a second set of risers adjoin the PCB but are not electrically coupled to the PCB.

Clause 12: The analyte sensor system of Clause 11, wherein: the first set of risers and the first set of floating point pads are configured to shape a radiation pattern of the stamp antenna; and the second set of risers are configured to provide structural support to the stamp antenna.

Clause 13: The analyte sensor system of any one of Clauses 10-12, wherein: the analyte sensor system includes one or more other hardware components; and the one or more other hardware components are disposed on the bottom side of the PCB in the area between the main body portion of the stamp antenna and the bottom side of the PCB.

Clause 14: The analyte sensor system of Clause 13, wherein the one or more other hardware components include at least one of: a processor; a memory; an accelerometer; a force sensor; or a temperature sensor.

Clause 15: The analyte sensor system of Clauses 1-14, further comprising one or more loop antennas.

Clause 16: The analyte sensor system of Clause 15, wherein: the stamp antenna is configured for Bluetooth communication; and the one or more loop antennas are configured for NFC communication.

Clause 17: The analyte sensor system of Clause 16, wherein: the one or more loop antennas are configured to receive a request for the data indicative of the measured analyte levels using NFC communication; and the analyte sensor system is configured to transmit the data indicative of the measured analyte levels via the stamp antenna using Bluetooth communication based on the request.

Clause 18: A method for wireless communication by an analyte sensor system, comprising: obtaining, from an analyte sensor of the analyte sensor system, electrical current indicative of analyte levels of a user of the analyte sensor system; processing the electrical current to generate data indicative of the analyte levels; and transmitting the data indicative of the analyte levels using a stamp antenna, wherein the stamp antenna is disposed on a bottom side of printed circuit board (PCB) of the analyte sensor system configured to face a body of the user when the analyte sensor system is worn by the user.

Clause 19: The method of Clause 18, further comprising, when transmitting the data indicative of the analyte levels using the stamp antenna, applying a voltage to an exciter device disposed on a top side of the analyte sensor system configured to face away from the body of the user when the analyte sensor system is worn by the user.

Clause 20: The method of Clause 19, wherein transmitting the data indicative of the analyte levels using the stamp antenna comprises radiating energy from the stamp antenna including the data indicative of the analyte levels.

Clause 21: The method of Clause 20, wherein the analyte sensor system further includes a ground plane disposed on the top side of the PCB.

Clause 22: The method of Clause 21, wherein applying the voltage to the exciter device when transmitting the data indicative of the analyte levels causes the energy radiated from the stamp antenna to couple with the ground plane disposed on the top side of the PCB and to be re-radiated by the ground plane away from the body of the user.

Clause 23: The method of any one of Clauses 21-22, wherein applying the voltage to the exciter device when transmitting the data indicative of the analyte levels causes a majority of the energy radiated in a main lobe of the stamp antenna to be radiated away from the body of the user in a direction of the ground plane disposed on the top side of the PCB.

Clause 24: An apparatus for wireless communication by an analyte sensor system, comprising: one or more processors configured to execute instructions stored on one or more memories and to cause the analyte sensor system to: obtain, from an analyte sensor of the analyte sensor system, electrical current indicative of analyte levels of a user of the analyte sensor system; process the electrical current to generate data indicative of the analyte levels; and transmit the data indicative of the analyte levels using a stamp antenna, wherein the stamp antenna is disposed on a bottom side of printed circuit board (PCB) of the analyte sensor system configured to face a body of the user when the analyte sensor system is worn by the user.

Clause 25: The apparatus of Clause 24, wherein, when transmitting the data indicative of the analyte levels using the stamp antenna, the one or more processors are further configured to cause the analyte system to apply a voltage to an exciter device disposed on a top side of the analyte sensor system configured to face away from the body of the user when the analyte sensor system is worn by the user.

Clause 26: The apparatus of Clause 25, wherein, in order to transmit the data indicative of the analyte levels using the stamp antenna, the one or more processors are configured to cause the analyte sensor system to radiate energy from the stamp antenna including the data indicative of the analyte levels.

Clause 27: The apparatus of Clause 26, wherein the analyte sensor system further includes a ground plane disposed on the top side of the PCB.

Clause 28: The apparatus of Clause 27, wherein, when applying the voltage to the exciter device when transmitting the data indicative of the analyte levels, the one or more processors are further configured to cause the energy radiated from the stamp antenna to couple with the ground plane disposed on the top side of the PCB and to be re-radiated by the ground plane away from the body of the user.

Clause 29: The apparatus of any one of Clauses 27-28, wherein, when applying the voltage to the exciter device when transmitting the data indicative of the analyte levels, the one or more processors are further configured to cause a majority of the energy radiated in a main lobe of the stamp antenna to be radiated away from the body of the user in a direction of the ground plane disposed on the top side of the PCB.

Clause 30: An antenna system of an analyte sensor system for communicating data indicative of measured analyte levels of a user of the analyte sensor system, comprising: a stamp antenna configured to transmit the data indicative of the measured analyte levels; a ground plane configured to: obtain energy radiated from the stamp antenna including the data indicative of the measured analyte levels; and re-radiate the energy, including the data indicative of the measured analyte levels, away from the body of the user; and an exciter device configured to couple the energy radiated from the stamp antenna to the ground plane, wherein: the stamp antenna is disposed on a bottom side of a printed circuit board (PBC); the bottom side of the PCB is configured to face a body of the user when the analyte sensor system is worn by the user; and the ground plane and the exciter device are disposed on a top side of the PCB opposite to the bottom side of the PCB.

Clause 31: The antenna system of Clause 30, wherein: the stamp antenna is further configured to obtain the data indicative of the measured analyte levels from a transceiver of the analyte sensor system; and in order to transmit the data indicative of the measured analyte levels, the stamp antenna is configured to radiate energy including the data indicative of the measured analyte levels.

Clause 32: The antenna system of Clause 31, wherein the exciter device is configured to cause a majority of the energy radiated in a main lobe of the stamp antenna to be radiated away from the body of the user in a direction of the ground plane disposed on the top side of the PCB.

Clause 33: The antenna system of any one of Clauses 30-32, wherein: the stamp antenna includes a main body portion and plurality of risers coupled to the main body portion; and the plurality of risers are configured to raise the main body portion of the stamp antenna off of the bottom side of the PCB by a certain distance and form an area between the main body portion of the stamp antenna and the bottom side of the PCB.

Clause 34: The antenna system of Clause 33, wherein: a first set of risers are electrically coupled to a first set of floating point pads disposed on the bottom side of the PCB; and a second set of risers adjoin the PCB but are not electrically coupled to the PCB.

Clause 35: The antenna system of Clause 34, wherein: the first set of risers and the first set of floating point pads are configured to shape a radiation pattern of the stamp antenna; and the second set of risers are configured to provide structural support to the stamp antenna.

Clause 36: The antenna system of any one of Clauses 30-35, further comprising one or more loop antennas.

Clause 37: The antenna system of Clause 36, wherein: the stamp antenna is configured for Bluetooth communication; and the one or more loop antennas are configured for NFC communication.

Clause 38: The antenna system of Clause 37, wherein: the one or more loop antennas are configured to receive a request for the data indicative of the measured analyte levels using NFC communication; and the stamp antenna is configured to transmit the data indicative of the measured analyte levels using Bluetooth communication based on the request.

### Additional Considerations

In this document, the terms “computer program medium” and “computer usable medium” and “computer readable medium”, as well as variations thereof, are used to generally refer to transitory or non-transitory media. These and other various forms of computer program media or computer usable/readable media may be involved in carrying one or more sequences of one or more instructions to a processing device for execution. Such instructions embodied on the medium, may generally be referred to as “computer program code” or a “computer program product” or “instructions” (which may be grouped in the form of computer programs or other groupings). When executed, such instructions may enable a computing module, such as the analyte sensor system 208 and/or analyte sensor system 700, circuitry related thereto, and/or a processor thereof or connected thereto to perform features or functions of the present disclosure as discussed herein (for example, in connection with methods described above and/or in the claims), including for example when the same is/are incorporated into a system, apparatus, device and/or the like.

Various embodiments have been described with reference to specific example features thereof. It will, however, be evident that various modifications and changes may be made thereto without departing from the broader spirit and scope of the various embodiments as set forth in the appended claims. The specification and figures are, accordingly, to be regarded in an illustrative rather than a restrictive sense. It will be appreciated that, for clarity purposes, the above description has described embodiments with reference to different functional units. However, it will be apparent that any suitable distribution of functionality between different functional units may be used without detracting from the invention. For example, functionality illustrated to be performed by separate computing devices may be performed by the same computing device. Likewise, functionality illustrated to be performed by a single computing device may be distributed amongst several computing devices. Hence, references to specific functional units are only to be seen as references to suitable means for providing the described functionality, rather than indicative of a strict logical or physical structure or organization.

Although described above in terms of various example embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the present application, whether or not such embodiments are described and whether or not such features are presented as being a part of a described embodiment. Thus, the breadth and scope of the present application should not be limited by any of the above-described example embodiments.

Terms and phrases used in the present application, and variations thereof, unless otherwise expressly stated, should be construed as open ended as opposed to limiting. As examples of the foregoing: the term “including” should be read as meaning “including, without limitation” or the like; the term “example” is used to provide illustrative instances of the item in discussion, not an exhaustive or limiting list thereof; the terms “a” or “an” should be read as meaning “at least one,” “one or more” or the like; the term “set” should be read to include one or more objects of the type included in the set; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Similarly, the plural may in some cases be recognized as applicable to the singular and vice versa. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic, circuitry, or other components, may be combined in a single package or separately maintained and may further be distributed in multiple groupings or packages or across multiple locations.

Additionally, the various embodiments set forth herein are described in terms of example block diagrams, flow charts, and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration. Moreover, the operations and sub-operations of various methods described herein are not necessarily limited to the order described or shown in the figures, and one of skill in the art will appreciate, upon studying the present disclosure, variations of the order of the operations described herein that are within the spirit and scope of the disclosure. It will be understood that each block of the flowchart illustrations, and combinations of blocks in the flowchart illustrations, may be implemented by execution of computer program instructions. These computer program instructions may be loaded onto a computer or other programmable data processing apparatus (such as a controller, microcontroller, microprocessor or the like) in a sensor electronics system to produce a machine, such that the instructions which execute on the computer or other programmable data processing apparatus create instructions for implementing the functions specified in the flowchart block or blocks. These computer program instructions may also be stored in a computer-readable memory that may direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks presented herein.

It should be appreciated that all methods and processes disclosed herein may be used in any glucose or other analyte monitoring system, continuous or intermittent. It should further be appreciated that the implementation and/or execution of all methods and processes may be performed by any suitable devices or systems, whether local or remote. Further, any combination of devices or systems may be used to implement the present methods and processes.

In addition, the operations and sub-operations of methods described herein may be carried out or implemented, in some cases, by one or more of the components, elements, devices, modules, circuitry, processors, etc. of systems, apparatuses, devices, environments, and/or computing modules described herein and referenced in various of figures of the present disclosure, as well as one or more sub-components, elements, devices, modules, processors, circuitry, and the like depicted therein and/or described with respect thereto. In such instances, the description of the methods or aspects thereof may refer to a corresponding component, element, etc., but regardless of whether an explicit reference is made, one of skill in the art will recognize upon studying the present disclosure when the corresponding component, element, etc. may be used. Further, it will be appreciated that such references do not necessarily limit the described methods to the particular component, element, etc. referred to. Thus, it will be appreciated by one of skill in the art that aspects and features described above in connection with (sub-) components, elements, devices, modules, and circuitry, etc., including variations thereof, may be applied to the various operations described in connection with methods described herein, and vice versa, without departing from the scope of the present disclosure.

What is claimed is:

1. An analyte sensor system, comprising:
an analyte sensor configured to measure analyte levels of a user of the analyte sensor system;
a stamp antenna configured to transmit data indicative of the measured analyte levels;
a printed circuit board (PCB) that operatively connects the analyte sensor to the stamp antenna for transmission of the data indicative of the measured analyte levels; and
a housing that encases at least the stamp antenna, the PCB, and a first portion of the analyte sensor, wherein:
the housing has a bottom portion through which a second portion of the analyte sensor protrudes to an exterior of the housing of the analyte sensor system;
the bottom portion of the housing is configured to be attached to a body of the user; and
the stamp antenna is disposed on a bottom side of the PCB facing the bottom portion of the housing.

2. The analyte sensor system of claim 1, further comprising:
an exciter device disposed on a top side of the PCB opposite to the bottom side of the PCB; and
a ground plane disposed on the top side of the PCB.

3. The analyte sensor system of claim 2, further comprising:
a transceiver; and
one or more processors configured to:
obtain the measured analyte levels from the analyte sensor; and
output the data indicative of the measured analyte levels to the transceiver for transmission via the stamp antenna.

4. The analyte sensor system of claim 3, wherein:
the stamp antenna is further configured to obtain the data indicative of the measured analyte levels from the transceiver of the analyte sensor system;
in order to transmit the data indicative of the measured analyte levels, the stamp antenna is configured to radiate energy including the data indicative of the measured analyte levels; and
the exciter device is configured to cause a majority of the energy radiated in a main lobe of the stamp antenna to be radiated away from the body of the user in a direction of the ground plane disposed on the top side of the PCB.

5. The analyte sensor system of claim 4, wherein:
the exciter device is configured to couple the radiated energy from the stamp antenna to the ground plane; and
based on the exciter device, the ground plane is configured to:
obtain the energy from the stamp antenna; and
re-radiate the energy including the data indicative of the measured analyte levels away from the body of the user.

6. The analyte sensor system of claim 1, wherein:
the first portion of the analyte sensor is electrically coupled to the PCB using a conductive epoxy and an encapsulant layer; and
the second portion of the analyte sensor is configured to be implanted into the body of the user.

7. The analyte sensor system of claim 1, wherein:
the stamp antenna includes a main body portion and plurality of risers coupled to the main body portion; and
the plurality of risers are configured to raise the main body portion of the stamp antenna off of the bottom side of the PCB by a certain distance and form an area between the main body portion of the stamp antenna and the bottom side of the PCB.

8. The analyte sensor system of claim 7, wherein:
a first set of risers are electrically coupled to a first set of floating point pads disposed on the bottom side of the PCB;
a second set of risers adjoin the PCB but are not electrically coupled to the PCB;
the first set of risers and the first set of floating point pads are configured to shape a radiation pattern of the stamp antenna; and
the second set of risers are configured to provide structural support to the stamp antenna.

9. The analyte sensor system of claim 7, wherein:
the analyte sensor system includes one or more other hardware components;
the one or more other hardware components are disposed on the bottom side of the PCB in the area between the main body portion of the stamp antenna and the bottom side of the PCB; and
the one or more other hardware components include at least one of:
a processor;
a memory;
an accelerometer;
a force sensor; or
a temperature sensor.

10. The analyte sensor system of claim 1, further comprising one or more loop antennas, wherein:
the stamp antenna is configured for Bluetooth communication;
the one or more loop antennas are configured for NFC communication;
the one or more loop antennas are configured to receive a request for the data indicative of the measured analyte levels using NFC communication; and
the analyte sensor system is configured to transmit the data indicative of the measured analyte levels via the stamp antenna using Bluetooth communication based on the request.

11. A method for wireless communication by an analyte sensor system, comprising:
obtaining, from an analyte sensor of the analyte sensor system, electrical current indicative of analyte levels of a user of the analyte sensor system;
processing the electrical current to generate data indicative of the analyte levels; and
transmitting the data indicative of the analyte levels using a stamp antenna, wherein the stamp antenna is disposed on a bottom side of printed circuit board (PCB) of the analyte sensor system configured to face a body of the user when the analyte sensor system is worn by the user.

12. The method of claim 11, further comprising, when transmitting the data indicative of the analyte levels using the stamp antenna, applying a voltage to an exciter device disposed on a top side of the analyte sensor system configured to face away from the body of the user when the analyte sensor system is worn by the user.

13. The method of claim 12, wherein transmitting the data indicative of the analyte levels using the stamp antenna comprises radiating energy from the stamp antenna including the data indicative of the analyte levels.

14. The method of claim 13, wherein the analyte sensor system further includes a ground plane disposed on the top side of the PCB.

15. The method of claim 14, wherein applying the voltage to the exciter device when transmitting the data indicative of the analyte levels causes the energy radiated from the stamp antenna to couple with the ground plane disposed on the top side of the PCB and to be re-radiated by the ground plane away from the body of the user.

16. The method of claim 14, wherein applying the voltage to the exciter device when transmitting the data indicative of the analyte levels causes a majority of the energy radiated in a main lobe of the stamp antenna to be radiated away from the body of the user in a direction of the ground plane disposed on the top side of the PCB.

17. An apparatus for wireless communication by an analyte sensor system, comprising:
one or more processors configured to execute instructions stored on one or more memories and to cause the analyte sensor system to:
obtain, from an analyte sensor of the analyte sensor system, electrical current indicative of analyte levels of a user of the analyte sensor system;
process the electrical current to generate data indicative of the analyte levels; and
transmit the data indicative of the analyte levels using a stamp antenna, wherein the stamp antenna is disposed on a bottom side of printed circuit board (PCB) of the analyte sensor system configured to face a body of the user when the analyte sensor system is worn by the user.

18. The apparatus of claim 17, wherein, when transmitting the data indicative of the analyte levels using the stamp antenna, the one or more processors are further configured to cause the analyte sensor system to apply a voltage to an exciter device disposed on a top side of the analyte sensor system configured to face away from the body of the user when the analyte sensor system is worn by the user.

19. The apparatus of claim 18, wherein:
in order to transmit the data indicative of the analyte levels using the stamp antenna, the one or more processors are configured to cause the analyte sensor system to radiate energy from the stamp antenna including the data indicative of the analyte levels;
the analyte sensor system further includes a ground plane disposed on the top side of the PCB; and
when applying the voltage to the exciter device when transmitting the data indicative of the analyte levels, the one or more processors are further configured to cause the energy radiated from the stamp antenna to couple with the ground plane disposed on the top side of the PCB and to be re-radiated by the ground plane away from the body of the user.

20. The apparatus of claim 19, wherein, when applying the voltage to the exciter device when transmitting the data indicative of the analyte levels, the one or more processors are further configured to cause a majority of the energy radiated in a main lobe of the stamp antenna to be radiated away from the body of the user in a direction of the ground plane disposed on the top side of the PCB.
